(19) United States
(12) Patent Application Publication
(10) Pub. No.: US 2014/0121517 A1
(43) Pub. Date: May 1, 2014

(54) METHOD AND APPARATUS FOR MONITORING TEMPERATURE USING ULTRASOUND

(71) Applicant: Samsung Electronics Co., Ltd., Suwon-si (KR)

(72) Inventors: Sang-hyun KIM, Hwaseong-si (KR); Won-chul BANG, Seongnam-si (KR); Ki-wan CHOI, Anyang-si (KR); Young-kyoo HWANG, Seoul (KR); Ji-young PARK, Yongin-si (KR)

(73) Assignee: SAMSUNG ELECTRONICS CO., LTD., Suwon-si (KR)

(21) Appl. No.: 14/066,876

(22) Filed: Oct. 30, 2013

(30) Foreign Application Priority Data
Oct. 30, 2012 (KR) 10-2012-0121524

(51) Int. Cl. A61B 8/08; A61B 5/01

(52) U.S. Cl. CPC A61B 8/5223; A61B 5/01
USPC 600/438

(57) ABSTRACT

Provided are a method and apparatus for monitoring a temperature using an ultrasound wave. The method of monitoring a temperature using an ultrasound wave may include generating ultrasound speed data according to temperature in tissue of a target object, from ultrasound speed data according to temperature in each component forming the tissue, based on volume fraction of each of the components; and estimating a temperature of the tissue based on an ultrasound echo signal reflected by the tissue and the generated ultrasound speed data according to temperature.

### CROSS-REFERENCE TO RELATED APPLICATIONS

This application claims the benefit under 35 U.S.C. §119(a) of Korean Patent Application No. 10-2012-0121524, filed on Oct. 30, 2012, in the Korean Intellectual Property Office, the entire disclosures of which is hereby incorporated by reference for all purpose.

### BACKGROUND

1. Field

The following description relates to a method and apparatus for monitoring a temperature using an ultrasound wave and to a system for treatment and diagnosis using an ultrasound wave.

2. Description of Related Art

Along with the development of medical science, minimally invasive surgery, and furthermore, noninvasive surgery, has been recently used for local treatment of tumors. Of noninvasive surgery methods, High Intensity Focused Ultrasound (HIFU) therapy is widely used because harm to a human body can be prevented by use of a sound wave. The HIFU therapy is a treatment method that causes necrosis of a lesion tissue by focusing and irradiating a high intensity ultrasonic wave on a lesion inside a human body. The ultrasonic wave, which is focused and irradiated on the lesion tissue, is transduced into thermal energy to increase a temperature of the irradiated part, causing coagulation necrosis in the tissue and a blood vessel. Since the temperature may be instantaneously increased, only the irradiated part may be effectively removed while preventing thermal diffusion around the irradiated part.

When a lesion exists in tissue of a target object, a system for treatment and diagnosis using an ultrasound wave makes a burn by irradiating an ultrasound wave for treatment on the lesion through an ultrasound treatment device and diagnoses whether a therapy has been completed by acquiring ultrasound images of tissue with the lesion through an ultrasound diagnosis device. In addition, by monitoring a temperature change in the tissue with the lesion, whether a therapy has been completed may be perceived.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided a method of monitoring a temperature using an ultrasound wave, the method including generating ultrasound speed data according to temperature in tissue of a target object, from ultrasound speed data according to temperature in each component forming the tissue, based on volume fraction of each of the components; and estimating a temperature of the tissue based on an ultrasound echo signal reflected by the tissue and the generated ultrasound speed data according to temperature.

The generating of ultrasound speed data according to temperature may include generating ultrasound speed data according to temperature for each unit volume of the tissue based on volume fraction of the components for each unit volume of the tissue.

The unit volume may be a voxel.

The generating of ultrasound speed data according to temperature in the tissue may include analyzing volume fraction of the components forming the tissue for each unit volume of the tissue from pre-diagnosis information of the tissue; transforming the analyzed volume fraction of the components based on the pre-diagnosis information into volume fraction of the components for each unit volume of the target object in an ultrasound image; and acquiring the ultrasound speed data according to temperature in the tissue by combining ultrasound speed data according to temperature in the components of the transformed volume fractions.

The transforming may include matching a position of the target object in the pre-diagnosis information with a position of the target object in the ultrasound image; and obtaining volume fraction of the components for each unit volume of the tissue of the target object in the matched ultrasound image by using the analyzed volume fraction of the components for each unit volume based on the pre-diagnosis information.

The transforming of the analyzed volume fraction of the components may include transforming the analyzed volume fraction of the components using interpolation or extrapolation.

The estimating of the temperature of the tissue may include measuring a speed value from the echo signal reflected by irradiating the ultrasound wave on the tissue; comparing the generated ultrasound speed data according to temperature with the measured speed value; and determining a temperature corresponding to the generated ultrasound speed data matching the measured speed value as a temperature of the tissue.

The method may include acquiring pre-diagnosis information of the tissue and the volume fraction of the components forming the tissue are analyzed from the acquired pre-diagnosis information.

The pre-diagnosis information of the tissue may be at least one of magnetic resonance imaging (MRI) data, magnetic resonance spectroscopy data, or computed tomography (CT) data.

The method may include generating a temperature image of the tissue by using the estimated temperature.

A non-transitory computer-readable recording medium storing a computer-readable program for executing the method.

In another general aspect, there is provided an apparatus for monitoring a temperature using an ultrasound wave, the apparatus including a speed data generator configured to generate ultrasound speed data according to temperature in tissue of a target object, from ultrasound speed data according to temperature in each component forming the tissue, based on volume fraction of the components; a measurer configured to measure a speed value from an ultrasound echo signal reflected by the tissue; and an estimator configured to estimate a temperature of the tissue by using the measured speed value and the generated ultrasound speed data according to temperature.

The speed data generator may be configured to generate ultrasound speed data according to temperature for each unit volume of the tissue based on volume fraction of the components for each unit volume of the tissue.

The unit volume may be a voxel.

The speed data generator may include an analyzer configured to analyze volume fraction of the components forming the tissue for each unit volume of the tissue from pre-diagnosis information of the tissue; a transformer configured to transform the analyzed volume fraction of the components based on the pre-diagnosis information into volume fraction of the components for each unit volume of the target object in an ultrasound image; and an acquirer configured to acquiring the ultrasound speed data according to temperature in the tissue by combining ultrasound speed data according to temperature in the components of the transformed volume fractions.

The transformer may be configured to match a position of the target object in the pre-diagnosis information with a position of the target object in the ultrasound image and obtains volume fraction of the components for each unit volume of the tissue of the target object in the matched ultrasound image by using the analyzed volume fraction of the components for each unit volume based on the pre-diagnosis information.

The estimator may be configured to compare the generated ultrasound speed data according to temperature with the measured speed value and to determine a temperature corresponding to the generated ultrasound speed data matching the measured speed value as a temperature of the tissue.

a storage for storing pre-diagnosis information of the tissue and ultrasound speed data according to temperature for the components forming the tissue.

The pre-diagnosis information of the tissue may be at least one of magnetic resonance imaging (MRI) data, magnetic resonance spectroscopy data, or computed tomography (CT) data.

The apparatus may include a temperature image generator configured to generate a temperature image of the tissue by using the estimated temperature.

In another general aspect, there is provided an apparatus for treatment and diagnosis using an ultrasound wave, the system including an ultrasound treatment device configured to irradiate an ultrasound wave for treatment on a treatment part; an ultrasound diagnosis device configured to irradiate an ultrasound wave for diagnosis on tissue of a target object including the treatment part and to receive an echo signal of the irradiated ultrasound wave for diagnosis; an image processing device configured to control the ultrasound treatment device and the ultrasound diagnosis device, acquire ultrasound speed data according to temperature in the tissue of the target object from ultrasound speed data according to temperature in each component forming the tissue based on volume fraction of the components, estimate a temperature of the tissue by using a speed value, which is measured from the received echo signal, and generate a temperature image of the tissue by using the estimated temperature; and an image display device configured to display the generated temperature image.

Other features and aspects will be apparent from the following detailed description, the drawings, and the claims.

### BRIEF DESCRIPTION OF THE DRAWINGS

FIG. 1 is a diagram illustrating an example of a system for treatment and diagnosis using an ultrasound wave.

FIG. 2 is a diagram illustrating an example of an apparatus for monitoring a temperature using an ultrasound wave.

FIG. 3 is a diagram illustrating examples of volume fractions of components forming tissue of a target object on which an ultrasound wave is irradiated.

FIGS. 4A to 4C are diagrams illustrating examples of ultrasound speed data according to temperatures in components forming tissue.

FIGS. 5A to 5C are diagrams illustrating examples of ultrasound speed data according to temperatures in tissues having different volume fractions of components forming the tissues.

FIG. 6 is a diagram illustrating an example of a method of monitoring a temperature using an ultrasound wave.

FIG. 7 is a diagram illustrating an example of S620 of FIG. 6.

FIG. 8 is a diagram illustrating an example of S720 of FIG. 7.

FIG. 9 is a diagram illustrating examples of S630 of FIG. 6.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

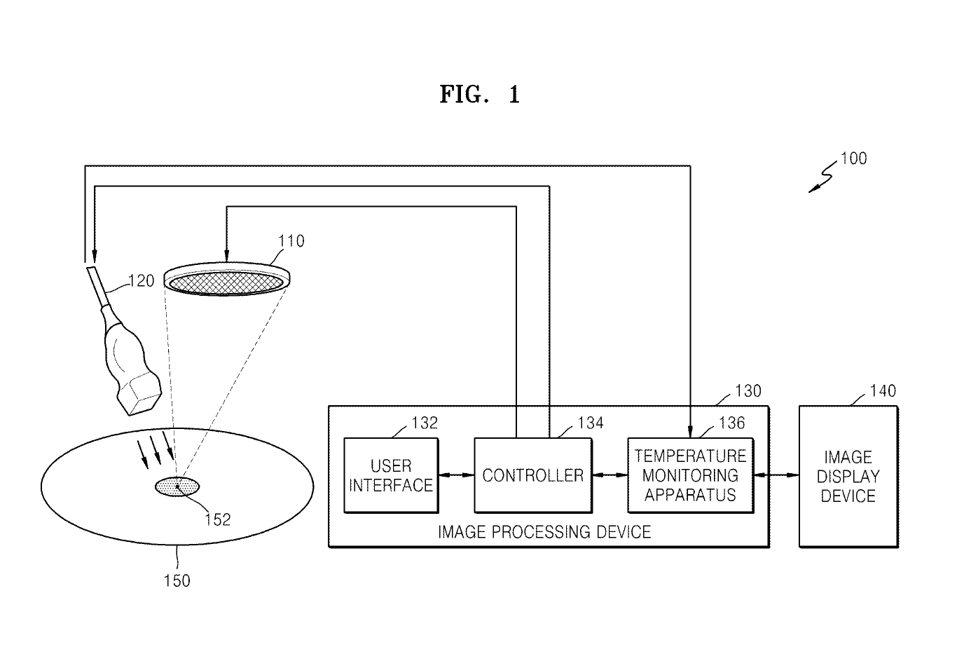

FIG. 1 is a diagram illustrating an example of a system 100 for treatment and diagnosis using an ultrasound wave. Referring to FIG. 1, the system 100 may include an ultrasound treatment device 110, an ultrasound diagnosis device 120, an image processing device 130, and an image display device 140. While components related to the present example are illustrated in the ultrasound system 100 of FIG. 1, it is understood that those skilled in the art may include other general components. In addition, the ultrasound treatment device 110, the ultrasound diagnosis device 120, the image processing device 130, and the image display device 140 forming the system 100 may not be physically separated as shown in FIG. 1, but may be integrated in one or more body.

When a lesion, such as a tumor, exists in a tissue 152 of a target object 150, the system 100 makes a burn by irradiating an ultrasound wave for treatment on the lesion through the ultrasound treatment device 110. The system 100 receives an echo signal reflected by irradiating an ultrasound wave for diagnosis on the tissue 152 with the lesion through the ultrasound diagnosis device 120, generates, and displays images of the tissue 152 with the lesion to help operators, such as medical practitioners, determine whether a therapy of the lesion has been completed. The ultrasound treatment device 110 irradiates the ultrasound wave for treatment towards the lesion to causes necrosis of a lesion tissue, thereby treating a patient. An HIUF may be used as the ultrasound wave for treatment.

The ultrasound diagnosis device 120, or a diagnosis probe, irradiates the ultrasound wave for diagnosis on the tissue 152 with the lesion and receives an echo signal obtained by reflecting the irradiated ultrasound wave for diagnosis. The ultrasound diagnosis device 120 irradiates the ultrasound wave for diagnosis on the tissue 152 with the lesion and receives a reflected echo signal to generate an ultrasound image of the tissue 152 with the lesion. In addition, the ultrasound diagnosis device 120 monitors a temperature change in the tissue 152 with the lesion by using the received echo signal. The echo signal may be used not only to generate an ultrasound image but also to monitor a temperature change in the tissue 152 with the lesion.

In the non-exhaustive example of FIG. 1, the ultrasound treatment device 110 and the ultrasound diagnosis device 120 are independent devices. However, the ultrasound treatment device 110 and the ultrasound diagnosis device 120 as separate modules in one device or as a single device are considered to be well within the scope of the present disclosure.

The image processing device 130 may receive a command from a user and may control the ultrasound treatment device 110 and the ultrasound diagnosis device 120. Moreover, the image processing device 130 generates images of the tissue 152 with the lesion by using echo signals received by the ultrasound diagnosis device 120. The image processing device 130 may include a user interface 132, a controller 134, and an apparatus 136 for monitoring a temperature.

The user interface 132 receives a command related to an operation of the system 100 from the user. The user interface 132 may also be responsible for inputting and outputting input information regarding a user and an image. The user interface 132 may include a network module for connection to a network and a universal serial bus (USB) host module for forming a data transfer channel with a mobile storage medium, depending on a function of the ultrasound system 100. In addition, the user interface 132 may include an input/output device such as, for example, a mouse, a keyboard, a touch screen, a monitor, a speaker, a screen, and a software module for running the input/output device.

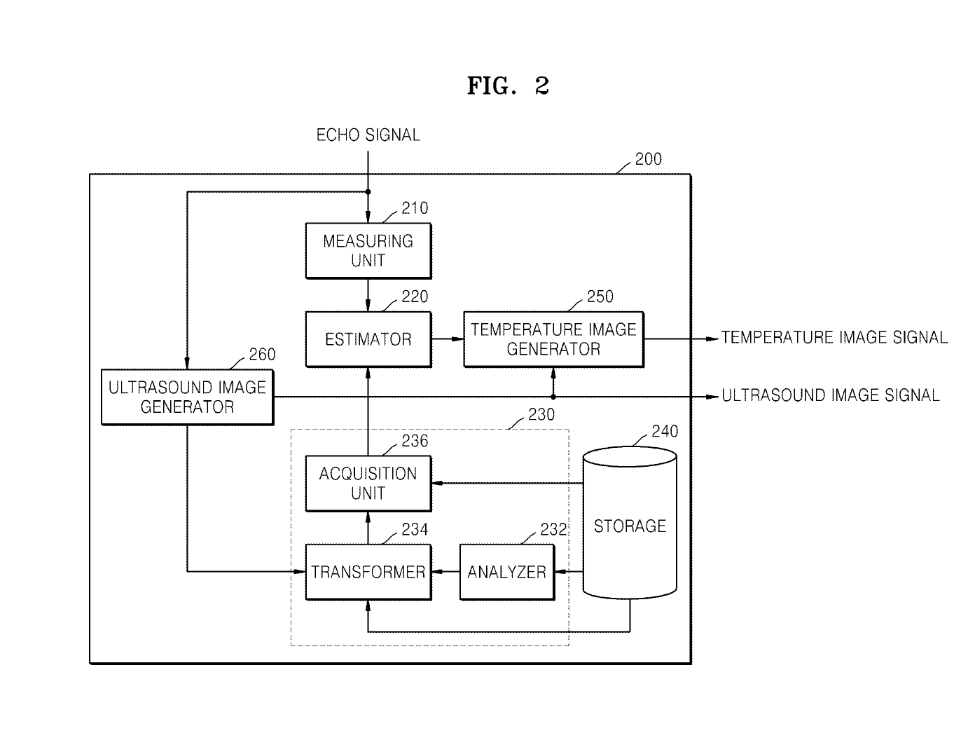

The controller 134 generates control signals to control operations of components in the image processing device 130 and to control the ultrasound treatment device 110 and the ultrasound diagnosis device 120. The apparatus 136 estimates a temperature of the tissue 152 with the lesion by using the echo signal of the ultrasound wave for diagnosis, generates a temperature image of the tissue 152 with the lesion by using the estimated temperature, and generates an ultrasound image of the tissue 152 with the lesion. Operation and function of the apparatus 136 according to a non-exhaustive example will be described below with reference to FIG. 2.

The image display device 140 receives signals related to generated images from the image processing device 130 and displays an ultrasound image, a temperature image, or the like, on a display (not shown). The display may be implemented as a liquid crystal display (LCD), a light-emitting diode (LED) display, a plasma display panel (PDP), a screen, a terminal, and the like. A screen may be a physical structure that includes one or more hardware components that provide the ability to render a user interface and/or receive user input. The screen can encompass any combination of display region, gesture capture region, a touch sensitive display, and/or a configurable area. The screen can be embedded in the hardware or may be an external peripheral device that may be attached and detached from the apparatus. The display may be a single-screen or a multi-screen display. A single physical screen can include multiple displays that are managed as separate logical displays permitting different content to be displayed on separate displays although part of the same physical screen.

FIG. 2 is a diagram illustrating an example of an apparatus 200 for monitoring a temperature using an ultrasound wave. Referring to FIG. 2, the apparatus 200 may include a measuring unit 210, an estimator 220, a speed data generator 230, a storage 240, a temperature image generator 250, and an ultrasound image generator 260. The speed data generator 230 may include an analyzer 232, a transformer 234, and an acquisition unit 236. While components related to the present example are illustrated in the ultrasound system 200 for monitoring a temperature of FIG. 2, it is understood that those skilled in the art may include other general components.

The apparatus 200 generates a temperature image by estimating a temperature of tissue using an echo signal of an ultrasound wave for diagnosis. In addition, the apparatus 200 may generate an ultrasound wave of the tissue.

When a temperature of the tissue 152 is monitored, the temperature is monitored with respect to a plurality of unit volumes to more accurately monitor the temperature. The tissue 152 is divided into a plurality of unit volumes to more accurately monitor the temperature. In a non-exhaustive example, the unit volume may be a voxel.

The measuring unit 210 measures a speed value from an echo signal reflected by irradiating an ultrasound wave on the tissue. The measuring unit 210 measures a speed value of an ultrasound wave reflected from each of the plurality of voxels forming the tissue.

The estimator 220 estimates a temperature of the tissue based on ultrasound speed data according to temperature values of the tissue, and the speed value of the ultrasound wave reflected from each of the plurality of voxels. The measuring unit 210 measures the speed value of the ultrasound wave reflected from each of the plurality of voxels. The estimator 220 compares the ultrasound speed data according to temperature values of the tissue with the measured speed value of the ultrasound wave reflected from each of the plurality of voxels. Based on the result of the comparison, the estimator 220 determines a temperature that matches each ultrasound speed data as a temperature of each voxel of the tissue.

The ultrasound speed data according to temperature values of the tissue indicates ultrasound speed data reflected from each unit volume, i.e., each voxel, and a corresponding temperature. The ultrasound speed data according to temperatures in the tissue is generated by the speed data generator 230 to indicate that a target object on which an ultrasound wave is irradiated, i.e., biological tissue, is heterogeneous tissue comprising a plurality of components. Ultrasound speed data according to temperatures in tissue including one component is known to one of ordinary skill in the art. However, when ultrasound speed data according to temperatures in one component is used for monitoring temperature, it is assumed that biological tissue includes one component, thereby causing an error in temperature monitoring. To more accurately monitor a temperature, ultrasound speed data according to temperatures in heterogeneous tissue is generated. The operation of the speed data generator 230 will now be described for generating ultrasound speed data according to temperatures in heterogeneous tissue.

The speed data generator 230 generates ultrasound speed data according to temperatures in tissue of a target object, such as, for example, biological tissue, on which an ultrasound wave is irradiated from previously-known ultrasound speed data according to temperatures in each component of volume fractions of components forming the tissue. The ultrasound speed data according to temperatures in the tissue may be acquired by calculating ultrasound speed data according to temperatures in each of the voxels forming the tissue. Since the tissue is heterogeneous, each of the voxels forming the tissue also has heterogeneous characteristics, and the components forming the tissue may have different volume fractions in each voxel. Thus, the ultrasound speed data according to temperatures in each voxel is generated for volume fractions of the components in each voxel. Referring to FIG. 2, the speed data generator 230 includes three sub-modules, such as the analyzer 232, the transformer 234, and the acquisition unit 236.

The analyzer 232 evaluates the volume fractions of the components for each of the voxels of the tissue in advance by acquiring the pre-diagnosis information of the tissue of the target object on which an ultrasound wave is irradiated. The pre-diagnosis information of the tissue may be data such as, for example, magnetic resonance data, such as magnetic resonance imaging (MRI) data, magnetic resonance spectroscopy data, or computed tomography (CT) data.

For example, from the MRI data, an image of fat or water may be obtained by mathematically adding or subtracting data of two images obtained using Out of Phase and In Phase protocols, and volume fractions of fat and water forming the tissue may be obtained using the image of fat or water. As another example, volume fractions of the components for each of the voxels forming the tissue may be obtained using the magnetic resonance spectroscopy data. However, the volume fractions of the components for each of the voxels forming the tissue, which are obtained from the pre-diagnosis information, such as MRI data, magnetic resonance spectroscopy data, or CT data, pertain to tissue in the pre-diagnosis information. It may be inappropriate to apply the volume fractions of the components obtained from the pre-diagnosis information to tissue in an ultrasound image. The volume fractions of the components for each of the voxels forming the tissue, which are analyzed by the analyzer 232, are volume fractions for each voxel in a magnetic resonance domain, magnetic resonance spectroscopy domain, or CT domain. But, when a temperature is monitored using an ultrasound wave, the volume fractions of the components for each voxel forming the tissue is in an ultrasound domain. The transformer 234 transforms the volume fractions of the components for each of the voxels forming the tissue based on the pre-diagnosis information into volume fractions of the components for each voxel in an ultrasound image for the same tissue.

Matching a position of the target object in the pre-diagnosis information with a position of the target object in the ultrasound image may need to be performed in advance. Since voxel coordinates in the pre-diagnosis information are different from voxel coordinates in the ultrasound image, matching is performed for coordinates in the two domains to correspond to each other, and an image matching method or a matching method using a marker may be used. An example of such a method of matching between two images is disclosed by W. H. Nam, D. G. Kang, D. Lee, and J. B. Ra in “Automatic registration between 3D intra-operative ultrasound and pre-operative CT images of the liver based on robust edge matching,” Physics in Medicine and Biology, 57, 69-91, 201, which is incorporated by reference. Thus, volume fractions of the components for each voxel of the tissue of the target object in the matched ultrasound image may be obtained using an interpolation or extrapolation method.

The acquisition unit 236 acquires ultrasound speed data according to temperatures in heterogeneous tissue by using the ultrasound speed data according to temperatures in each component, and the volume fractions of the components for each voxel of the tissue of the target object in the ultrasound image, which are obtained by the transformer 234. The acquisition unit 236 acquires ultrasound speed data according to temperatures in the tissue by combining the ultrasound speed data according to temperatures in the components according to the transformed volume fractions of the components obtained by the transformer 234. An example of the operation of the acquisition unit 236 will be described below.

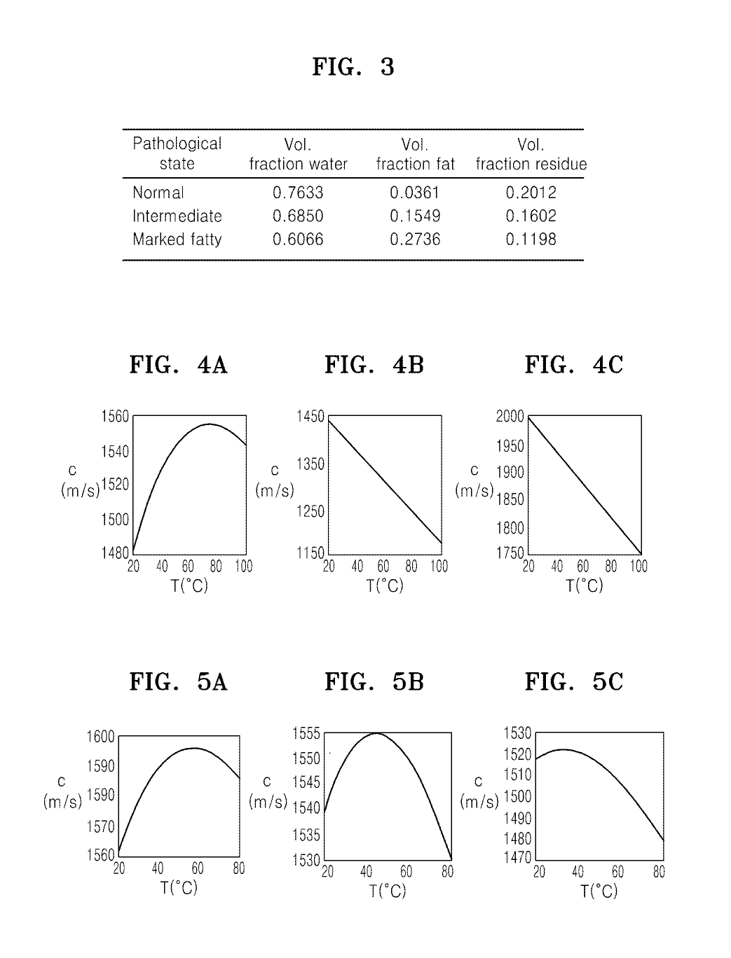

FIG. 3 is a diagram illustrating an example of volume fractions of components forming tissue of a target object on which an ultrasound wave is irradiated. FIG. 3 shows volume fractions of the components for some voxels forming the tissue of the target object in an ultrasound image obtained by the transformer 234. FIG. 3 shows volume fractions of water, fat, and the residue for three voxels. The first row of the table shows volume fractions of the components in a voxel having a normal tissue state, the third row shows volume fractions in a voxel having a fatty tissue state, and the second row shows volume fractions in a voxel having an intermediate state between normal tissue and fatty tissue. If it is assumed that the volume fractions of water, fat, and the residue are x_{w}, x_{f}, and xᵣ, respectively, the sum of x_{w}, x_{f}, and xᵣ in each voxel is 1.

FIGS. 4A to 4C are diagrams showing ultrasound speed data according to temperatures in components forming tissue. FIG. 4A is a graph showing ultrasound speed data according to temperatures in water, FIG. 4B is a graph showing ultrasound speed data according to temperatures in fat, and FIG. 4C is a graph showing ultrasound speed data according to temperatures in the residue. The ultrasound speed data according to temperatures in water, fat, and the residue are already known.

If it is assumed that the ultrasound speed data according to temperatures in water, fat, and the residue are c_{w}(T), c_{f}(T), and cᵣ(T) and the volume fractions of water, fat, and the residue are x_{w}, x_{f}, and xᵣ, respectively, ultrasound speed data according to temperatures in tissue including water, fat, and the residue may be obtained by Equation 1.

$$\frac{1}{c ( T )} = \frac{{x}_{w}}{{c}_{w} ( T )} + \frac{{x}_{f}}{{c}_{f} ( T )} + \frac{{x}_{r}}{{c}_{r} ( T )} \qquad Equation 1$$

FIGS. 5A to 5C are graphs obtained using Equation 1, which show voxels having different volume fractions of components. FIGS. 5A to 5C show ultrasound speed data according to temperatures in a voxel having a normal tissue state, a voxel having an intermediate state between normal tissue and fatty tissue, and a voxel having a fatty tissue state, respectively.

FIGS. 5A to 5C are diagrams showing ultrasound speed data according to temperatures in tissues having different volume fractions of components forming the tissues. FIG. 5A is a graph showing ultrasound speed data according to temperatures in the voxel having the normal tissue state. FIG. 5B is a graph showing ultrasound speed data according to temperatures in the voxel having the intermediate state between normal tissue and fatty tissue. FIG. 5C is a graph showing ultrasound speed data according to temperatures in the voxel having the fatty tissue state.

Referring back to FIG. 2, the storage 240 stores the pre-diagnosis information of the tissue, such as, for example, the MRI data, the magnetic resonance spectroscopy data, or the CT data. In addition, the storage 240 stores the ultrasound speed data according to temperatures for the components forming the tissue. For example, the storage 240 stores the ultrasound speed data according to temperatures in water, fat, and the residue as shown in FIGS. 4A to 4C.

The temperature image generator 250 generates a temperature image of the tissue by using the temperature estimated by the estimator 220. The temperature image is an image in which a temperature distribution of the tissue of the target object on which an ultrasound wave is irradiated is displayed with different colors or different brightnesses. The temperature distribution of the tissue of the target object may be generated by mapping the estimated temperature to an ultrasound image of the tissue. Thus, a temperature at a certain point of the tissue may be easily perceived. A temperature image signal indicating the temperature image is input to the image display device 140 and is displayed on the display unit of the image display device 140.

The ultrasound image generator 260 generates ultrasound images using echo signals using a process that is known to one of ordinary skill in the art. In the example shown in FIG. 2, the ultrasound image generator 260 is included in the apparatus 200. In another non-exhaustive example, the ultrasound image generator 260 may exist outside the apparatus 200. When the ultrasound image generator 260 is disposed outside the apparatus 200, a temperature image signal indicating the temperature image is input to the image display device 140 and is displayed on the display unit of the image display device 140.

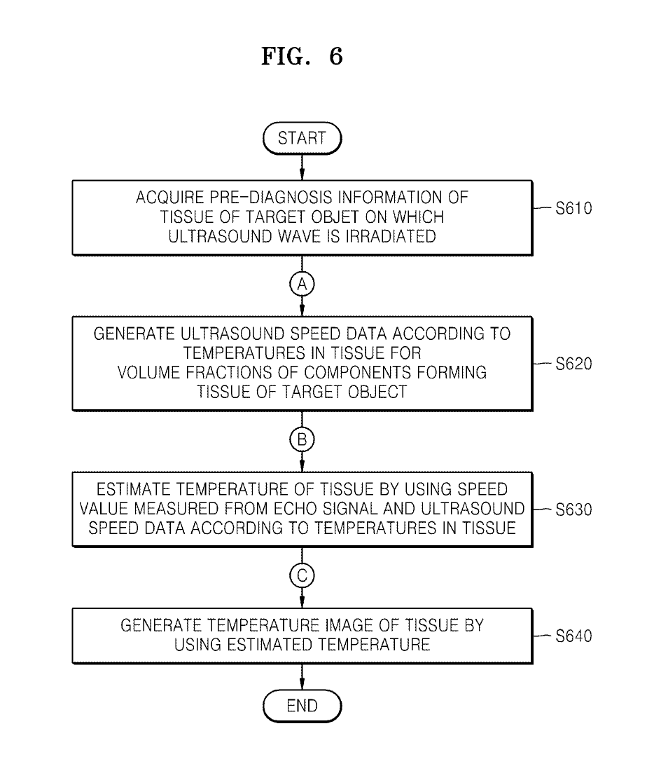

FIG. 6 is a diagram illustrating an example of a method of monitoring a temperature using an ultrasound wave. The operations in FIG. 6 may be performed in the sequence and manner as shown, although the order of some operations may be changed or some of the operations omitted without departing from the spirit and scope of the illustrative examples described. Many of the operations shown in FIG. 6 may be performed in parallel or concurrently. The above descriptions of FIGS. 1-5C, is also applicable to FIG. 6, and is incorporated herein by reference. Thus, the above description may not be repeated here.

Referring to FIG. 6, in S610, pre-diagnosis information of tissue of a target object on which an ultrasound wave is irradiated is acquired. The pre-diagnosis information may be input from the outside or stored in advance in the storage 240 of apparatus 200. The pre-diagnosis information of the tissue may be data such as, for example, magnetic resonance data, such as MRI data or magnetic resonance spectroscopy data, or CT data.

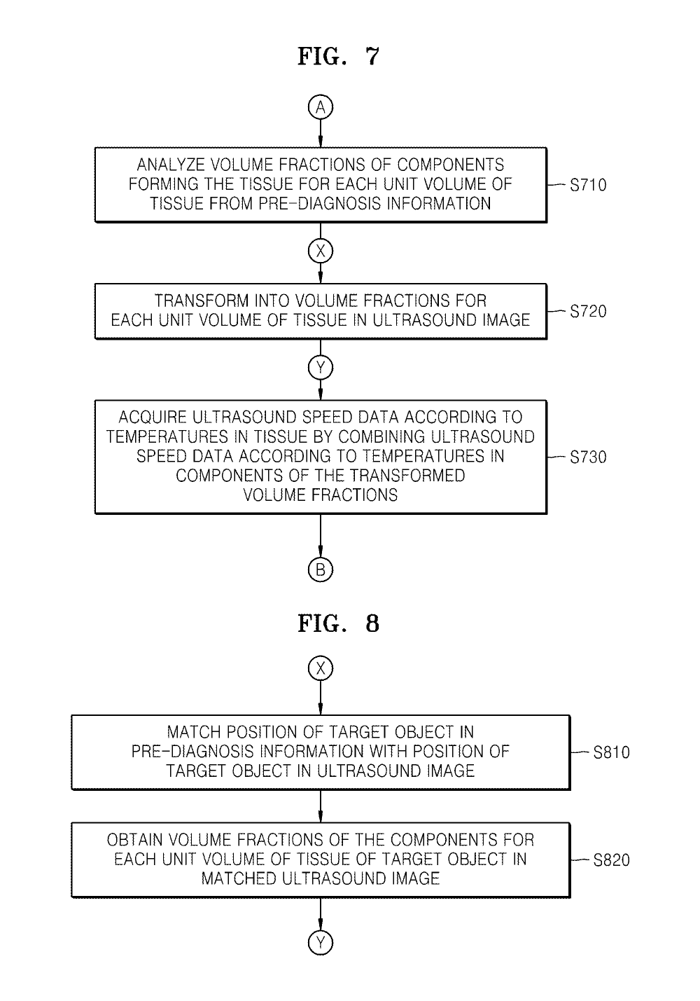

In S620, the speed data generator 230 generates ultrasound speed data according to temperatures in the tissue from ultrasound speed data according to temperatures in each component of the components forming the tissue of the target object on which an ultrasound wave is irradiated. For better temperature monitoring, ultrasound speed data according to temperatures for each unit volume of the tissue is generated for volume fractions of the components for each unit volume of the tissue. In this case, the unit volume may be a voxel, and the volume fractions of the components forming the tissue may be obtained from the pre-diagnosis information of the tissue. S620 will be described in more detail with reference to FIGS. 7 and 8.

FIG. 7 is a diagram illustrating an example of S620 of FIG. 6. The operations in FIG. 7 may be performed in the sequence and manner as shown, although the order of some operations may be changed or some of the operations omitted without departing from the spirit and scope of the illustrative examples described. Many of the operations shown in FIG. 7 may be performed in parallel or concurrently. The above descriptions of FIGS. 1-6, is also applicable to FIG. 7, and is incorporated herein by reference. Thus, the above description may not be repeated here.

Referring to FIG. 7, in S710, the analyzer 232 analyzes volume fractions of the components forming the tissue for each unit volume of the tissue from the pre-diagnosis information of the tissue of the target object. For example, when the pre-diagnosis information is magnetic resonance data, volume fractions analyzed from the magnetic resonance data correspond to volume fractions of the components forming the tissue in a magnetic resonance domain.

In S720, the transformer 234 transforms the volume fractions analyzed based on the pre-diagnosis information into volume fractions for each unit volume of the same tissue of the target object in an ultrasound image generated from an echo signal of an ultrasound wave for diagnosis. S720 will be described in more detail with reference to FIG. 8.

FIG. 8 is a diagram illustrating an example of S720 of FIG. 7. The operations in FIG. 8 may be performed in the sequence and manner as shown, although the order of some operations may be changed or some of the operations omitted without departing from the spirit and scope of the illustrative examples described. Many of the operations shown in FIG. 8 may be performed in parallel or concurrently. The above descriptions of FIGS. 1-7, is also applicable to FIG. 8, and is incorporated herein by reference. Thus, the above description may not be repeated here. Referring to FIG. 8, the operation of transforming into volume fractions for each unit volume of the same tissue of the target object in an ultrasound image may be divided into two operations.

In S810, the transformer 234 matches a position of the target object in the pre-diagnosis information with a position of the same target object in the ultrasound image. Since voxel coordinates of the target object in the pre-diagnosis information are different from voxel coordinates of the same target object in the ultrasound image, matching is performed for coordinates in the two domains to correspond to each other.

In S820, the transformer 234 obtains volume fractions of the components for each unit volume of the tissue of the target object in the matched ultrasound image by using the analyzed volume fractions for the pre-diagnosis information. The volume fractions of the components for each voxel of the tissue of the target object in the matched ultrasound image may be obtained by interpolating or extrapolating the volume fractions for each unit volume based on the pre-diagnosis information.

Referring back to FIG. 7, in S730, the acquisition unit 236 acquires ultrasound speed data according to temperatures in the tissue by combining ultrasound speed data according to temperatures in the components of the volume fractions transformed by the transformer 234.

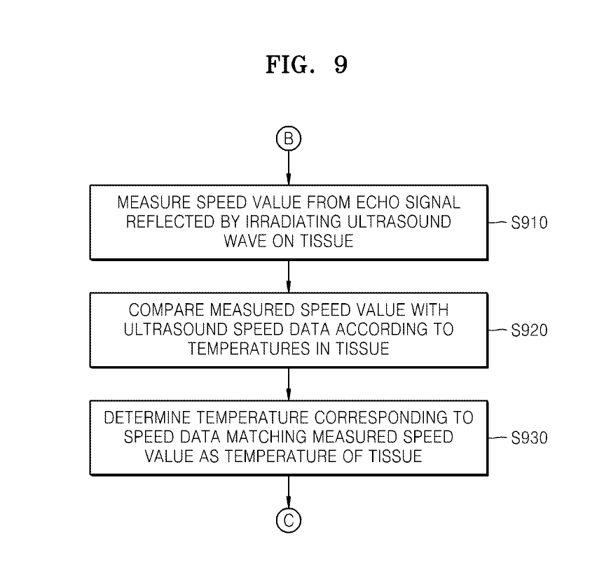

Referring back to FIG. 6, in S630, the estimator 220 estimates a temperature of the tissue by using a speed value, which is measured from an echo signal reflected by irradiating an ultrasound wave on the tissue, and the ultrasound speed data according to temperatures in the tissue, which is generated by the speed data generator 230. S630 will be described in more detail with reference to FIG. 9.

FIG. 9 is a diagram illustrating an example of S630 of FIG. 6. The operations in FIG. 9 may be performed in the sequence and manner as shown, although the order of some operations may be changed or some of the operations omitted without departing from the spirit and scope of the illustrative examples described. Many of the operations shown in FIG. 9 may be performed in parallel or concurrently. The above descriptions of FIGS. 1-8, is also applicable to FIG. 9, and is incorporated herein by reference. Thus, the above description may not be repeated here.

Referring to FIG. 9, in S910, the measuring unit 210 measures a speed value from the echo signal reflected by irradiating an ultrasound wave on the tissue. The measured speed value of the ultrasound wave is transmitted to the estimator 220 to be used to estimate a temperature of the tissue.

In S920, the estimator 220 compares the ultrasound speed data according to temperatures in the tissue, which is generated by the speed data generator 230, with the speed value measured by the measuring unit 210.

In S930, the estimator 220 determines a temperature corresponding to speed data matching the measured speed value as a temperature of the tissue based on the comparison result of S920.

Referring back to FIG. 6, in S640, the temperature image generator 250 generates a temperature image of the tissue by using the temperature estimated by the estimator 220.

As described above, according to the one or more of the above non-exhaustive examples, a temperature of heterogeneous tissue consisting of a plurality of components may be monitored.

The methods described above can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, WiFi, etc.). In addition, functional programs, codes, and code segments for accomplishing the example disclosed herein can be construed by programmers skilled in the art based on the flow diagrams and block diagrams of the figures and their corresponding descriptions as provided herein.

The apparatuses and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of monitoring a temperature using an ultrasound wave, the method comprising:
generating ultrasound speed data according to temperature in tissue of a target object, from ultrasound speed data according to temperature in each component forming the tissue, based on volume fraction of each of the components; and
estimating a temperature of the tissue based on an ultrasound echo signal reflected by the tissue and the generated ultrasound speed data according to temperature.

2. The method of claim 1, wherein the generating of ultrasound speed data according to temperature comprises generating ultrasound speed data according to temperature for each unit volume of the tissue based on volume fraction of the components for each unit volume of the tissue.

3. The method of claim 2, wherein the unit volume is a voxel.

4. The method of claim 1, wherein the generating of ultrasound speed data according to temperature in the tissue comprises:
analyzing volume fraction of the components forming the tissue for each unit volume of the tissue from pre-diagnosis information of the tissue;
transforming the analyzed volume fraction of the components based on the pre-diagnosis information into volume fraction of the components for each unit volume of the target object in an ultrasound image; and
acquiring the ultrasound speed data according to temperature in the tissue by combining ultrasound speed data according to temperature in the components of the transformed volume fractions.

5. The method of claim 4, wherein the transforming comprises:
matching a position of the target object in the pre-diagnosis information with a position of the target object in the ultrasound image; and
obtaining volume fraction of the components for each unit volume of the tissue of the target object in the matched ultrasound image by using the analyzed volume fraction of the components for each unit volume based on the pre-diagnosis information.

6. The method of claim 4, wherein the transforming of the analyzed volume fraction of the components comprises transforming the analyzed volume fraction of the components using interpolation or extrapolation.

7. The method of claim 1, wherein the estimating of the temperature of the tissue comprises:
measuring a speed value from the echo signal reflected by irradiating the ultrasound wave on the tissue;
comparing the generated ultrasound speed data according to temperature with the measured speed value; and
determining a temperature corresponding to the generated ultrasound speed data matching the measured speed value as a temperature of the tissue.

8. The method of claim 1, further comprising acquiring pre-diagnosis information of the tissue and the volume fraction of the components forming the tissue are analyzed from the acquired pre-diagnosis information.

9. The method of claim 8, wherein the pre-diagnosis information of the tissue is at least one of magnetic resonance imaging (MRI) data, magnetic resonance spectroscopy data, or computed tomography (CT) data.

10. The method of claim 1, further comprising generating a temperature image of the tissue by using the estimated temperature.

11. A non-transitory computer-readable recording medium storing a computer-readable program for executing the method of claim 1.

12. An apparatus for monitoring a temperature using an ultrasound wave, the apparatus comprising:
a speed data generator configured to generate ultrasound speed data according to temperature in tissue of a target object, from ultrasound speed data according to temperature in each component forming the tissue, based on volume fraction of the components;
a measurer configured to measure a speed value from an ultrasound echo signal reflected by the tissue; and
an estimator configured to estimate a temperature of the tissue by using the measured speed value and the generated ultrasound speed data according to temperature.

13. The apparatus of claim 12, wherein the speed data generator is configured to generate ultrasound speed data according to temperature for each unit volume of the tissue based on volume fraction of the components for each unit volume of the tissue.

14. The apparatus of claim 13, wherein the unit volume is a voxel.

15. The apparatus of claim 12, wherein the speed data generator comprises:
an analyzer configured to analyze volume fraction of the components forming the tissue for each unit volume of the tissue from pre-diagnosis information of the tissue;
a transformer configured to transform the analyzed volume fraction of the components based on the pre-diagnosis information into volume fraction of the components for each unit volume of the target object in an ultrasound image; and
an acquirer configured to acquiring the ultrasound speed data according to temperature in the tissue by combining ultrasound speed data according to temperature in the components of the transformed volume fractions.

16. The apparatus of claim 15, wherein the transformer is configured to match a position of the target object in the pre-diagnosis information with a position of the target object in the ultrasound image and obtains volume fraction of the components for each unit volume of the tissue of the target object in the matched ultrasound image by using the analyzed volume fraction of the components for each unit volume based on the pre-diagnosis information.

17. The apparatus of claim 12, wherein the estimator is configured to compare the generated ultrasound speed data according to temperature with the measured speed value and to determine a temperature corresponding to the generated ultrasound speed data matching the measured speed value as a temperature of the tissue.

18. The apparatus of claim 12, further comprising a storage for storing pre-diagnosis information of the tissue and ultrasound speed data according to temperature for the components forming the tissue.

19. The apparatus of claim 18, wherein the pre-diagnosis information of the tissue is at least one of magnetic resonance imaging (MRI) data, magnetic resonance spectroscopy data, or computed tomography (CT) data.

20. The apparatus of claim 12, further comprising a temperature image generator configured to generate a temperature image of the tissue by using the estimated temperature.

21. An apparatus for treatment and diagnosis using an ultrasound wave, the system comprising:
an ultrasound treatment device configured to irradiate an ultrasound wave for treatment on a treatment part;
an ultrasound diagnosis device configured to irradiate an ultrasound wave for diagnosis on tissue of a target object including the treatment part and to receive an echo signal of the irradiated ultrasound wave for diagnosis;
an image processing device configured to
control the ultrasound treatment device and the ultrasound diagnosis device,
acquire ultrasound speed data according to temperature in the tissue of the target object from ultrasound speed data according to temperature in each component forming the tissue based on volume fraction of the components,
estimate a temperature of the tissue by using a speed value, which is measured from the received echo signal, and
generate a temperature image of the tissue by using the estimated temperature; and
an image display device configured to display the generated temperature image.
